(19) United States
(12) Patent Application Publication
(10) Pub. No.: US 2003/0040730 A1
(43) Pub. Date: Feb. 27, 2003

(54) Secondary attachment system for personal care article

(76) Inventors: Yvette L. Hammonds, Oshkosh, WI (US); Allan J. Krueger, Winneconne, WI (US); Shelley R. Rasmussen, Oshkosh, WI (US)

Correspondence Address: KIMBERLY-CLARK WORLDWIDE, INC., 401 NORTH LAKE STREET, NEENAH, WI, 54956

(21) Appl. No.: 10/037,278

(22) Filed: Dec. 20, 2001

Related U.S. Application Data
(60) Provisional application No. 60/313,604, filed on Aug. 20, 2001.

(51) Int. Cl. A61F 13/15

(52) U.S. Cl. 604/385.04

(57) ABSTRACT

An article (e.g., a sanitary napkin, incontinence pad or similar personal care product) that is to be worn with an undergarment having an attachment system that includes: a pair of wings including selectively releasable, interengaging fasteners such that the wings are adapted to hold the article to an undergarment; and a fastener component including a plurality of engagement members so it is adapted to engage the fabric of an undergarment. Desirably, the attachment system includes: a first wing extending from the first longitudinal edge of the sanitary napkin and a second wing extending from the second longitudinal edge of the article, each wing having a fixed end and a free end; a first fastener component comprising at least a portion of the first wing and the second wing, the first fastener component including an engagement section having a plurality engagement members; and a cooperating fastener component comprising at least a portion of the first wing and the second wing such that the first and second wings of the article are capable of being joined by movement of the respective components together generally along an attachment direction into an overlapping and interengaging configuration.

### FIELD OF THE INVENTION

The present invention relates to fastening systems for garments and other articles. More particularly, the present invention relates to interlocking, mechanical-type fastening systems which can be employed with disposable articles, such as gowns, diapers, incontinence garments and the like.

### BACKGROUND

Absorbent personal care articles such as sanitary napkins, incontinence pads and the like may be secured to an undergarment to hold the article in proper position during use. These articles frequently employ wings or flaps as well as garment attachment adhesive on the garment facing side of the article to secure the article to the undergarment. The wings or flaps typically employ adhesive to secure the end of the wing or flap to the undergarment.

Even with the combination of wings/flaps and garment attachment adhesive, users may encounter difficulty keeping the article in position. This problem is particularly apparent when the user is physically active or when heat and/or humidity weaken the adhesive used to secure the article.

While different types of attachment systems such as belts, snaps or the like are known, these systems increase the cost and complexity of the article as well as difficulty for the user to attach or apply the absorbent article.

### SUMMARY

The present invention addresses the problems described above by providing an article (e.g., a sanitary napkin, incontinence pad or similar personal care product) that is to be worn with an undergarment, the article having an attachment system that includes:

a pair of wings including selectively releasable, interengaging fasteners such that the wings are adapted to hold the article to an undergarment; and

a fastener component including a plurality of engagement members so it is adapted to engage the fabric of an undergarment.

Generally speaking, the attachment system includes:

a first wing extending from the first longitudinal edge of the sanitary napkin and a second wing extending from the second longitudinal edge of the article, each wing having a fixed end and a free end;

a first fastener component comprising at least a portion of the first wing and the second wing, the first fastener component including an engagement section having a plurality engagement members; and

a cooperating fastener component comprising at least a portion of the first wing and the second wing such that the first and second wings of the article are capable of being joined by movement of the respective components together generally along an attachment direction into an overlapping and interengaging configuration.

Importantly, the first fastener component is configured on the first wing and the second wing so that the overlapped and interengaged wings are adapted to secure the article to an undergarment, and an unengaged first fastener component is positioned adjacent the backsheet so it is adapted to engage the fabric of an undergarment positioned between the backsheet and the wings. This unengaged first fastener component would be located or positioned adjacent the backsheet in the absence of an undergarment between the backsheet and wings.

The first fastener component has a plurality of engagement members (also referred to as hook elements). Desirably, the first fastener component has a plurality of substantially non-isotropic engagement members such that the engagement section has an axis of substantially maximal engagement and the first fastener component is oriented so its axis of substantially maximal engagement is generally orthogonal to the attachment direction.

According to an aspect of the invention, each substantially non-isotropic engagement member has a stem portion with a distal end region and a securement element disposed at the distal end region of its corresponding stem portion.

The second fastener component is a loop material. Desirably, the loop material is a nonwoven loop material. More desirably, the nonwoven loop material is a pattern unbonded material. For example, the nonwoven loop material may be a pattern unbonded material such as, for example, the material described in U.S. Pat. Nos. 5,858,515 and/or 5,763,041, the contents of which are incorporated herein by reference.

In an embodiment of the invention, the fastening system can be used to secure the article in a configuration convenient for disposal.

The article may include additional fastening features such as, for example, garment attachment adhesive on the backsheet layer.

An embodiment of the invention is directed to a sanitary napkin having an attachment system that includes:

a pair of wings including selectively releasable, interengaging fasteners such that the wings are adapted to hold the sanitary napkin to an undergarment; and

a fastener component including a plurality of engagement members so it is adapted to engage the fabric of an undergarment.

Generally speaking, the sanitary napkin has a pair of end edges, a first longitudinal edge and a second longitudinal edge located between the end edges, a backsheet layer, a substantially liquid permeable topsheet layer and an absorbent disposed between the topsheet layer and the backsheet layer. The multiple attachment system is a selectively releasable, interengaging fastening system that includes:

a first wing extending from the first longitudinal edge of the sanitary napkin and a second wing extending from the second longitudinal edge of the sanitary napkin, each wing having a fixed end and a free end;

a first fastener component comprising at least a portion of the first wing and the second wing, the first fastener component including an engagement section having a plurality of engagement members; and

a cooperating fastener component comprising at least a portion of the first wing and the second wing such that the first and second wings of the sanitary napkin are capable of being joined by movement of the respective components together generally along an attachment direction into an overlapping and interengaging configuration.

Importantly, the first fastener component is configured on the first wing and the second wing so the overlapped and interengaged wings are adapted to secure the sanitary napkin to an undergarment, and further present an unengaged first fastener component adjacent the backsheet so it is adapted to engage the fabric of an undergarment positioned between the backsheet and the wings.

Another embodiment of the invention relates to a sanitary napkin having a pair of end edges, a first longitudinal edge and a second longitudinal edge located between the end edges, a backsheet layer, a substantially liquid permeable topsheet layer and an absorbent disposed between the topsheet layer and the backsheet layer; the sanitary napkin having a multiple attachment system that includes:

a first wing extending from the first longitudinal edge of the sanitary napkin and a second wing extending from the second longitudinal edge of the sanitary napkin, each wing having a fixed end and a free end;

a first fastener component including at least a portion of a bottom surface of the first wing and the second wing, the first fastener component including an engagement section having a plurality of engagement members; and

a cooperating fastener component including at least a portion of a top surface the first wing and the second wing such that the first and second wings can be interengaged by bringing a top and bottom surface of respective wings into an overlapping configuration;

so that the interengaged wings are adapted to secure the sanitary napkin to an undergarment, and further present an unengaged first fastener component adjacent the backsheet so it is adapted to engage the fabric of an undergarment positioned between the backsheet and the wings.

Generally speaking, the wings of the sanitary napkin can be moved together into their overlapping configuration generally along an attachment direction. Is it desirable that the first fastener component has a plurality of substantially non-isotropic engagement members such that the engagement section has an axis of substantially maximal engagement. It is also desirable that the first fastener component is oriented so its axis of substantially maximal engagement is generally orthogonal to the attachment direction.

Desirably, the second fastener component is a nonwoven loop material. More desirably, the nonwoven loop material is a pattern unbonded material.

In an embodiment of the invention, the attachment system can be used to secure the sanitary napkin in a configuration convenient for disposal.

The sanitary napkin may further include garment attachment adhesive on the backsheet layer. It is also contemplated that at least one wing of the attachment may include perforation lines adjacent its fixed end to provide quick removal of the napkin by tearing the wing along the perforation lines.

An embodiment of the present invention encompasses a sanitary napkin having a pair of end edges, a first longitudinal edge and a second longitudinal edge located between the end edges, a backsheet layer, a substantially liquid permeable topsheet layer and an absorbent disposed between the topsheet layer and the backsheet layer;

the sanitary napkin further including a pair of wings, each wing extending a longitudinal edge of the sanitary napkin and each wing having a fixed end and a free end; each wing comprising:

a first fastener component on a lower surface of the wing, the first fastener component including an engagement section having a plurality engagement members; and

a cooperating fastener component on an upper surface of the wing such that the wings of the sanitary napkin are capable of being joined by bringing the top and bottom surface of respective wings into an overlapping and interengaging configuration; wherein the interengaged wings are adapted to secure the sanitary napkin to an undergarment and further present an unengaged first fastener component adjacent the backsheet so it is adapted to engage the fabric of an undergarment positioned between the backsheet and the wings.

### BRIEF DESCRIPTION OF THE DRAWINGS

The present invention will be more fully understood and further advantages will become apparent when reference is made to the following detailed description of the invention and the drawings, in which:

FIG. 1A is an illustration of an exemplary article incorporating an exemplary fastening system of the invention;

FIG. 1B is an illustration of an exemplary article incorporating an exemplary fastening system of the invention;

FIG. 1C is an illustration of an exemplary article incorporating an exemplary fastening system of the invention as it is used with an undergarment;

FIG. 1D is an illustration of an exemplary article incorporating an exemplary fastening system of the invention—with the backsheet of the article facing upward

FIG. 1E is an illustration of a portion of an exemplary article incorporating an exemplary fastening system of the invention—with the backsheet of the article facing upward;

FIG. 1F is an illustration of an exemplary multiple fastening system of the present invention;

FIG. 2A is an illustration of an exemplary, non-isotropic engagement member which can be employed with the present invention;

FIG. 2B is an illustration of an exemplary, non-isotropic engagement member which can be employed with the present invention;

FIG. 2B′ representatively shows a top view of the engagement member of FIG. 3B;

FIG. 2C is an illustration of an exemplary, non-isotropic engagement member which can be employed with the present invention;

FIG. 2C′ representatively shows a top view of the engagement member of FIG. 3C;

FIG. 2D is an illustration of an exemplary, non-isotropic engagement member which can be employed with the present invention;

FIG. 2E is an illustration of an exemplary, non-isotropic engagement member which can be employed with the present invention;

FIGS. 3A-B are graphical representations of data in Table 1;

FIGS. 4A-B are graphical representations of data in Table 1;

FIGS. 5A-B are photomicrographs of an exemplary, non-isotropic engagement member which can be employed with the present invention; and

FIGS. 6 and 7 are photomicrographs of exemplary cooperating fastener members (e.g., loop materials) that may be used with the present invention.

### DETAILED DESCRIPTION OF THE INVENTION

As used herein, the terms “nonwovens” and “nonwoven web” means a web having a structure of individual fibers or threads that are interlaid, but not in any identifiable, repeating pattern. Nonwoven webs have been, in the past, formed by a variety of processes such as, for example, meltblowing processes, spunbonding processes, and bonded carded web processes.

As used herein, the term “spunbond web” refers to a web formed by extruding a molten thermoplastic material as filaments from a plurality of fine, usually circular, capillaries with the diameter of the extruded filaments then being rapidly reduced, for example, by fluid-drawing or other well known spunbonding mechanisms. The production of spunbond nonwoven webs is illustrated in patents such as Appel, et al., U.S. Pat. No. 4,340,563.

The various aspects and embodiments of the invention will be described in the context of a disposable absorbent article, such as a sanitary napkin or a disposable incontinence article. It is, however, readily apparent that the present invention could also be employed with other articles, such as caps, gowns, covers and the like in which an article it comes in contact with is secured to a garment such as, for example, an undergarment. Typically, the disposable articles are intended for limited use and are not intended to be laundered or otherwise cleaned for reuse. A sanitary napkin, for example, is discarded after it has become soiled by the wearer.

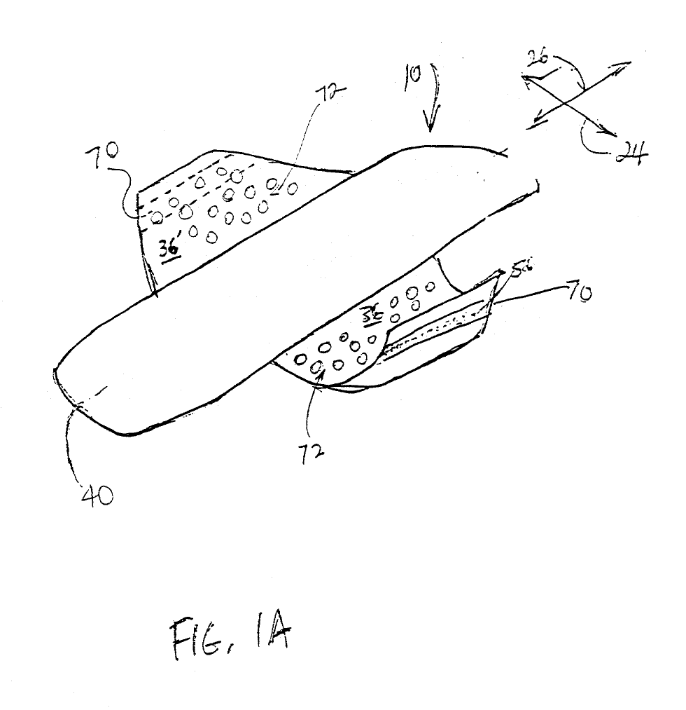

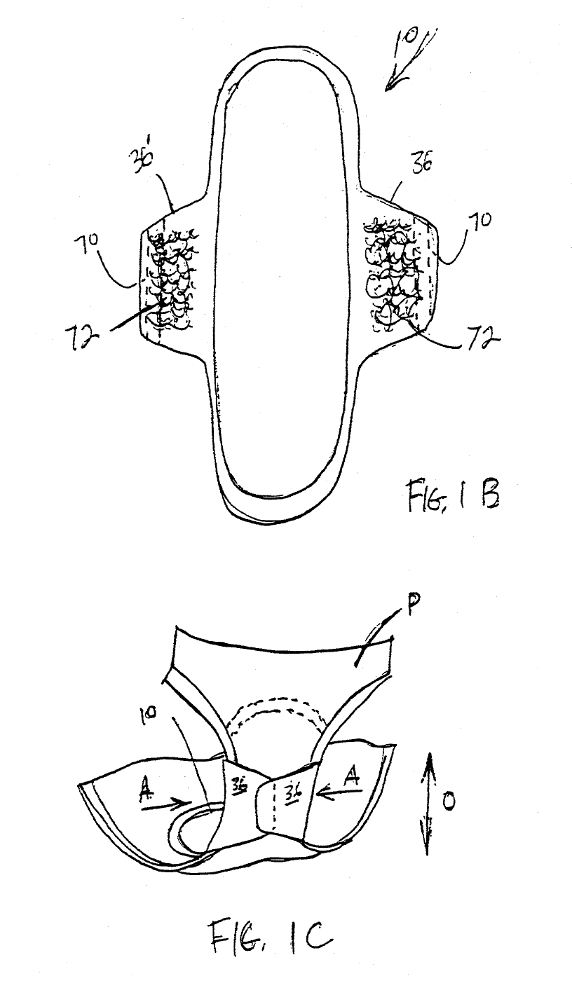

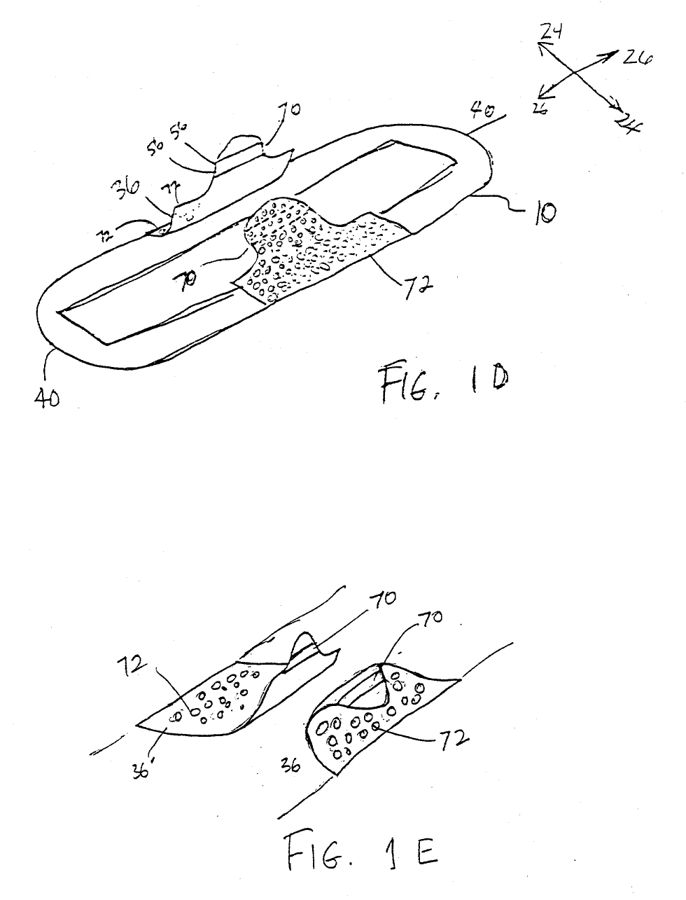

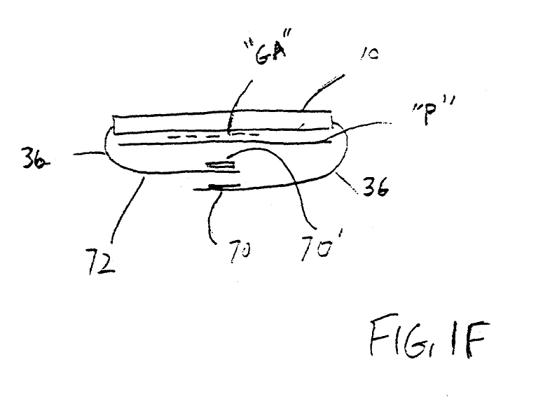

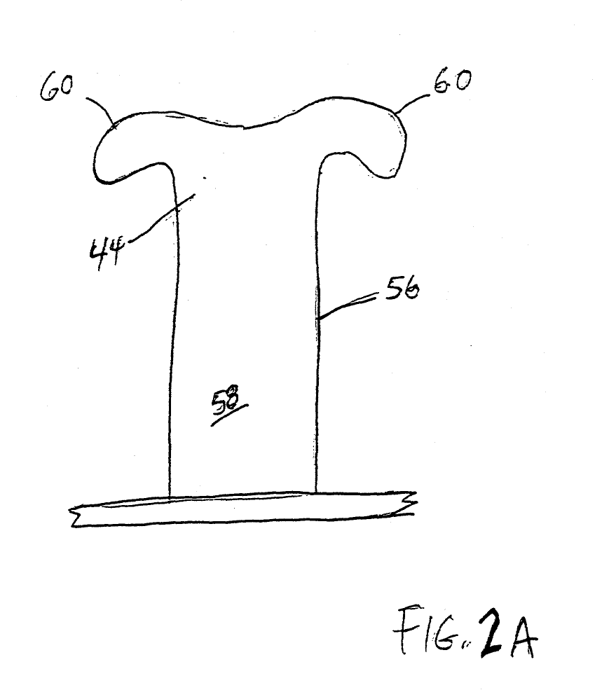

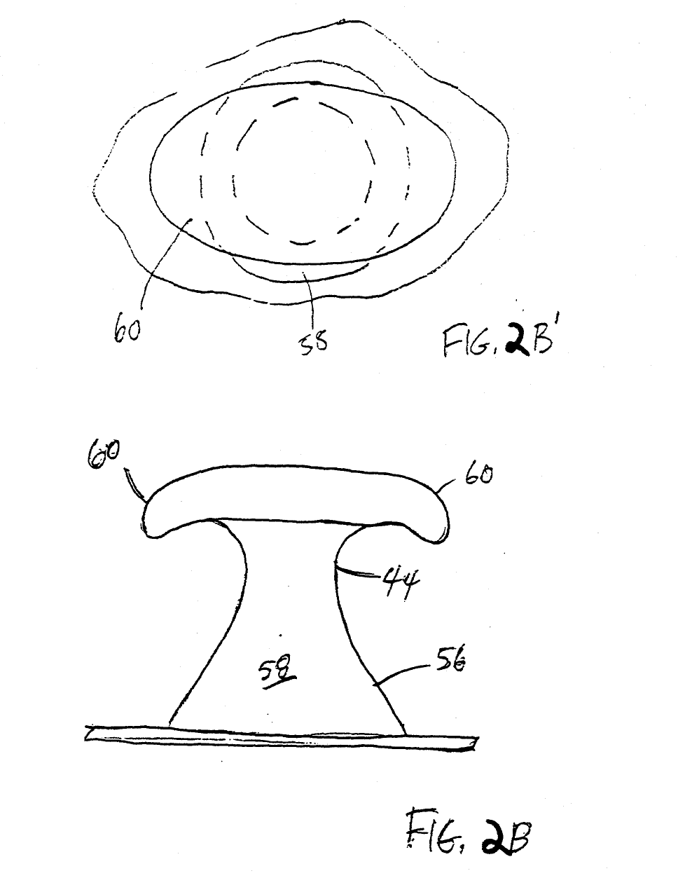

With reference to the Figures, an article, such as a sanitary napkin 10 illustrated in FIGS. 1A-D has a lengthwise, longitudinal direction 26, a lateral cross-direction 24, and a longitudinally extending medial line 40. The article includes a first article portion, a second article portion and at least one fastener 36 for securing the first article portion to the second article portion. Such securement can, for example, be configured to thereby hold the article on a wearer. The fastener includes at least one, first fastener component 70 attached to an appointed section of the first and second article portion, and a cooperating, second fastener component 72 attached to or integral with the first and second article portion. The first fastener component 70 includes an engagement section having a first plurality of engagement members 56. Each engagement member 56 has a stem portion 58 with a distal end region 44, and has at least one securement element 60 disposed at its corresponding distal end region. The plurality of engagement members has an arrangement pattern of their securement elements. It is contemplated that multiple pluralities of engagement members, each with different arrangement pattern of their securement elements, may be used.

Another aspect of the invention can provide an article in which the fastener component may include an engagement section having a plurality of non-isotropic engagement members. Each non-isotropic engagement member can have a stem portion 58 with a distal end portion 44, and a direction-dependent securement element 60 which is non-isotropically disposed at the distal end region of its corresponding stem portion 58 to provide a non-isotropic engagement opening. The plurality of non-isotropic engagement members can have an alignment pattern of their engagement openings. It is contemplated that multiple pluralities of non-isotropic engagement members may be used and that different alignment patterns of their engagement openings are possible.

In particular configurations, a majority of the plurality of non-isotropic engagement members have their engagement openings directed substantially orthogonal to an attachment direction. Generally speaking, the attachment direction is the direction in which the respective first and second portions of the article are brought together into an overlapping and interengaging relationship. Thus, in FIG. 1, the attachment direction is generally a direction having a cross-directional vector-component along the lateral direction 24 and toward the medial line 40 of the article. Accordingly, the plurality of non-isotropic engagement members would have their engagement openings directed substantially parallel with the medial line 40 of the article.

In the various aspects of the invention, the individual engagement members are typically flexible and resilient, but will substantially retain their initial shape during ordinary use. When flexed or deformed during ordinary use, the engagement members will substantially avoid plastically deforming to sustain the deformation, and will, instead, substantially return or “spring-back” to their original orientations and shape.

The various aspects (individually and in combination) of the present invention can advantageously help to better maintain the desired fit and position of the article when it is worn by a wearer. The incorporation of the various aspects of the fastening system of the invention can provide improved securement of an article with greater resistance to shifting out of position when the article is worn with an undergarment. By staying in position, the fastening system may also help provide improved fit, greater comfort and reduced irritation of the wearer's skin.

The article of the invention can, for example, be an absorbent article such as a sanitary napkin or incontinence article.

FIG. 1A is an illustration of an exemplary sanitary napkin with fasteners 36 in the form of wings or flaps. At least one first fastener component 70 is attached to the first wing 36 and second wing 36′ and at least one cooperating fastener component 72 is attached to or integral with the first wing 30 and second wing 36′. Importantly, the first fastener component 70 and the cooperating fastener component 72 are positioned on each wing such that the wings may be fastened without concern for overlapping the wings in any particular order. In some embodiments, the wings may be formed partially or entirely of the cooperating fastener component 72. FIG. 1C is an illustration of the sanitary napkin with its wings 36 or flaps secured around an undergarment or panty “P”. The arrows labeled “A” generally represent the attachment direction. The arrows labeled “O” generally represent the direction that is orthogonal to the attachment direction. It should be understood that this orthogonal direction is thought to be generally or substantially along or in the plane of the article although in some specific cases, it include a minor Z-direction component.

According to an embodiment of the invention, the first fastener component may be configured to have an axis of maximal engagement. This can be accomplished by utilizing a plurality of engagement members 56 that are non-isotropic (i.e., anisotropic) or non-symmetric. When the axis of maximal engagement of the first fastener component is oriented to be generally orthogonal to the attachment direction (that is, the direction each fastener component is generally brought together to effect overlapping engagement), it has been unexpectedly found that the fastening system is adapted to become more interengaged as the product is worn. For example, peel force and shear force as determined utilizing standard test procedures prior to wearing the article is greater for fastening systems in which the axis of maximal engagement of the first fastener component is oriented in the attachment direction and lower for fastening systems in which the axis of maximal engagement of the first fastener component is oriented orthogonal to the attachment direction.

When the peel force and the shear force were measured after use, the values increased for both orientations. However, the peel force and sheer force values measured for fastening systems in which the axis of maximal engagement of the first fastener component is oriented orthogonal to the attachment direction were greater than those measured for fastening systems in which the axis of maximal engagement of the first fastener component is oriented in the attachment direction.

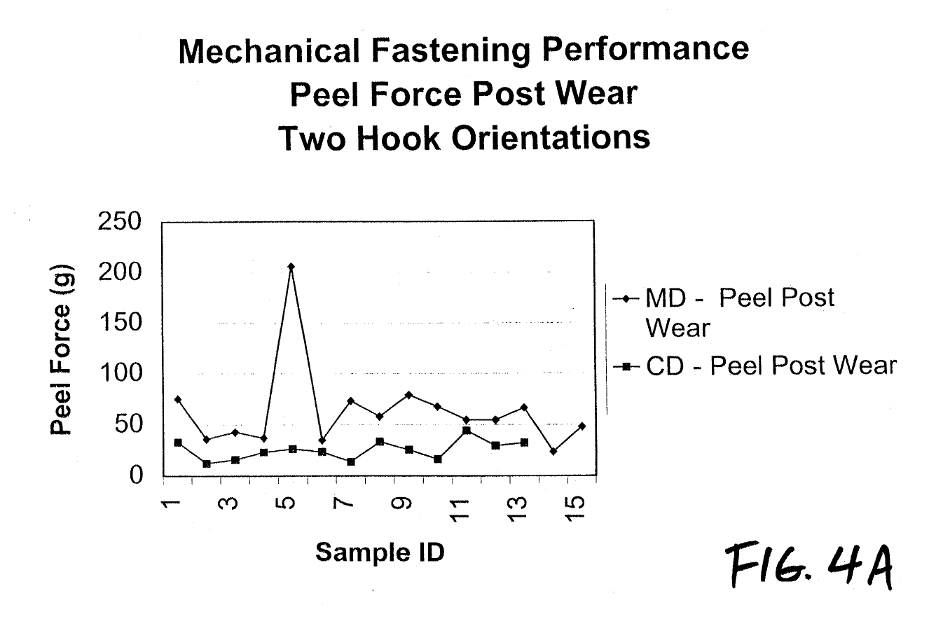

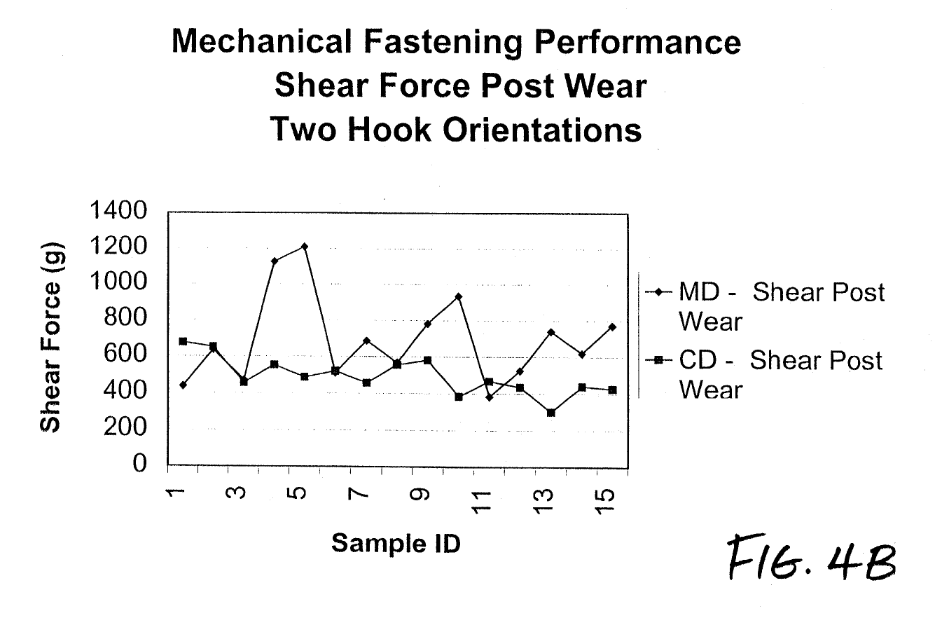

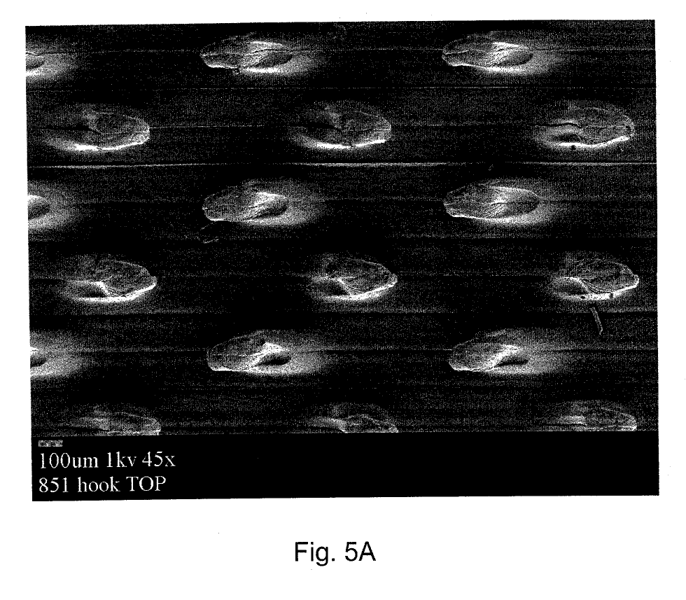

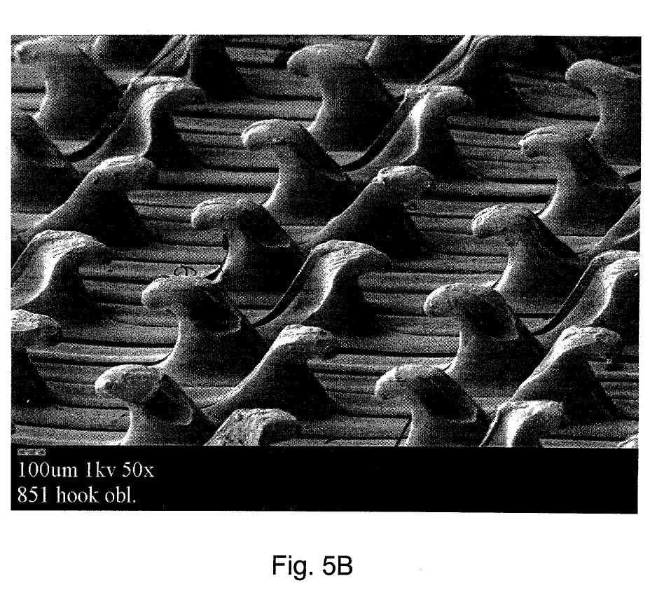

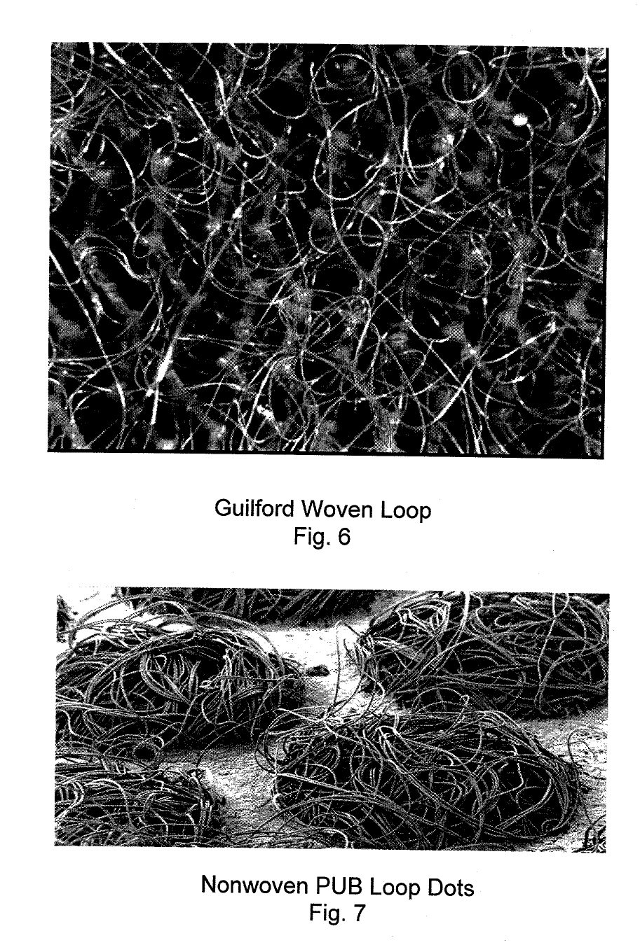

Data showing this result is shown graphically in FIGS. 3A-B and 4A-B and is included in Table 1 in the Examples section.

The following is a brief description of the orientation direction with respect to the lengthwise, longitudinal direction 26 and the lateral cross-wise direction depicted in FIG. 1. In one exemplary sanitary napkin, the orienting the axis of maximal engagement of the first fastener component in the attachment direction meant orienting the first fastener component so its axis of maximal engagement was in the cross-machine direction or the lateral cross-direction 24 shown in FIG. 1. Thus, for that sanitary napkin, the orienting the axis of maximal engagement of the first fastener component generally orthogonal to the attachment direction meant orienting the first fastener component so its axis of maximal engagement was in the machine direction or the lengthwise, longitudinal direction 26 shown in FIG. 1.

One feature of the present invention is evident from FIG. 1E which illustrates a portion of a multi-fastening system or secondary attachment system. FIG. 1E shows a pair of wings or flaps 36 and 36′ each having a first fastener component 70 affixed to the wing so as to face the baffle or peel strip of an article as well as a second fastener component 72 affixed to or integral with wing so as to face the opposite side of the wing (i.e., the side of the wind facing away from the baffle or peel strip).

When the wings of the article are secured around the crotch portion of an undergarment generally as depicted in FIG. 1C and as shown in cross-sectional view in FIG. 1F, the wings are adapted to hold, secure, attach or join the sanitary napkin or incontinence pad (or similar article) to an undergarment. In addition, a first fastener component including a plurality of engagement members is presented directly against the fabric of the undergarment such that they are adapted to engage the fabric of the undergarment to also help hold, secure, attach or join the sanitary napkin or incontinence pad (or similar article) to the undergarment.

More particularly, FIG. 1F shows such a system in which the fasteners or wings 36 that may be formed of the cooperating fastener component 72 are engaged by contact with the first fastener component 70. A strip of garment adhesive “GA” on the backsheet side or garment side of the article 10 can help secure the article to the underwear “P”. In addition, a first fastener component 70′ is in a position to engage the fabric of the underwear “P” while the fasteners or wings 36 secure or join the article to the underwear “P” by wrapping around the underwear.

With reference to the representative configurations shown in FIG. 1A-F, the article can include a system of flap regions, wings, “ear” regions or ear members. In particular arrangements, each flap, wing or ear region or member may extend laterally at the opposed, lateral (i.e., longitudinal) ends of the article such as an incontinence pad, sanitary napkin or the like.

In the various configurations of the invention, the ear, tab, flap or wing regions may be integrally formed with a selected article component. For example, ear, tab, flap or wing regions can be integrally formed from the layer of material that provides backsheet layer and/or may be integrally formed from the material employed to provide topsheet. In alternative configurations, the ear, tab, flap or wing regions may be provided by one or more separately provided members that are connected and assembled to the backsheet, to the topsheet, in between the backsheet and topsheet, or in various fixedly attached combinations of such assemblies.

In particular configurations of the invention, each of the ear, tab, flap or wing regions may be formed from a separately provided piece of material which is then suitably assembled and attached to a selected portion of the article.

The ear, tab, flap or wing regions may be composed of a substantially non-elastomeric material, such as polymer films, woven fabrics, nonwoven fabrics or the like, as well as combinations thereof. In particular aspects of the invention, ear, tab, flap or wing regions may be composed of a substantially elastomeric material, such as a stretch-bonded-laminate (SBL) material, a neck-bonded-laminate (NBL) material, an elastomeric film, an elastomeric foam material, or the like, which is elastomerically stretchable at least along the lateral direction 24. For example, suitable meltblown elastomeric fibrous webs for forming ear, tab, flap or wing regions are described in U.S. Pat. No. 4,663,220 issued May 5, 1987 to T. Wisneski et al., the entire disclosure of which is hereby incorporated by reference. Examples of composite fabrics comprising at least one layer of nonwoven textile fabric secured to a fibrous elastic layer are described in European Patent Application EP 0 217 032 A2 published on Apr. 8, 1987 which has the listed inventors of J. Taylor et al., the entire disclosure of which is hereby incorporated by reference. Examples of NBL materials are described in U.S. Pat. No. 5,226,992 issued Jul. 13, 1993 to Mormon, the entire disclosure of which is hereby incorporated by reference in a manner that is consistent herewith.

As previously mentioned, various suitable constructions can be employed to attach the ear, tab, flap or wing regions to the selected portions of the article. Particular examples of suitable constructions for securing a pair of elastically stretchable members to the lateral, side portions of an article to extend laterally outward beyond the laterally opposed side regions of the outer cover and liner components of an article can be found in U.S. Pat. No. 4,938,753 issued Jul. 3, 1990 to P. VanGompel et al., the entire disclosure of which is hereby incorporated by reference in a manner that is consistent herewith.

The illustrated ear, tab, flap or wing regions have a tapered, curved or otherwise contoured shape in which the longitudinal length of the relatively inboard base region is larger or smaller than the longitudinal length of its relatively outboard end region. Alternatively, the ear regions may have a substantially rectangular shape, and optionally may have a substantially trapezoidal shape.

In the various aspects and configurations of the invention, the fastening mechanism between the selected first fastener component and the selected, cooperating fastener component may be adhesive, cohesive, mechanical or combinations thereof. In the context of the present invention, a mechanical fastening system is a system which includes a first fastener component and cooperating fastener component which mechanically inter-engage to provide a desired securement.

Desirably, the first fastener component and cooperating fastener components include complementary elements of a cooperatively interengaging mechanical fastening system. The mechanical fastener components can be provided by mechanical-type fasteners such as hooks, buckles, snaps, buttons and the like, which include cooperating and complementary, mechanically interlocking components.

As shown in the illustrated arrangements, for example, the mechanical fastening system may be a hook-and-loop type of fastening system. Such fastening systems typically include engagement members having the form of a “hook” or hook-like, male component, and include a cooperating “loop” or loop-like, female component which engages and releasably interconnects with the hook component. Desirably, the interconnection is selectively releasable and re-attachable. Conventional systems are, for example, available under the VELCRO trademark. The hook element may be provided by a single-prong hook configuration, a multiple-prong hook configuration or by a generally continuous, expanded-head configuration, such as provided by a mushroom-head type of hook element. The loop element may be provided by a woven fabric, a nonwoven fabric, a knitted fabric, a perforated or apertured layer, and the like, as well as combinations thereof. The many arrangements and variations of such fastener systems have been collectively referred to as hook-and-loop fasteners.

A configuration which employs a selectively releasable, interengaging mechanical fastening system can, for example, locate the first fastener component 70 on at least the appointed mating or securing surface of the tabs, flaps or wings 36 and 36′, and can locate the cooperating, second fastener component on the appointed engagement surface of the appointed tabs, flaps or wings 36 and 36′. For example, with the representatively shown hook-and-loop fastener, the fastening component which is attached to the appointed mating or securing surface of the fastener tabs 36 and 36′ may include a hook type of mechanical engagement element, and the complementary fastening component, which is operably joined and attached to the appointed surface of the fastener tabs 36 and 36′ can include a loop type of fastening element.

It should also be readily apparent that, in the various configurations of the invention, the relative positions and/or materials of the first fastening components 70 and their cooperating, complementary fastening component 72 can be transposed.

Examples of hook-and-loop fastening systems and components are described in U.S. Pat. No. 5,019,073 issued May 28, 1991 to T. Roessler et al., the entire disclosure of which is hereby incorporated by reference in a manner that is consistent herewith. Other examples of hook-and-loop fastening systems are described in U.S. patent application Ser. No. 366,080 entitled HIGH-PEEL TAB FASTENER, filed Dec. 28, 1994 by G. Zehner et al. (attorney docket No. 11,571) which corresponds to U.S. Pat. No. 5,605,735; and U.S. patent application Ser. No. 421,640 entitled MULTI-ATTACHMENT FASTENING SYSTEM, filed Apr. 13, 1995 by P. VanGompel et al.; the entire disclosures of which are hereby incorporated by reference in a manner that is consistent herewith. Examples of fastening tabs constructed with a carrier layer are described in U.S. patent application Ser. No. 08/603,477 of A. Long et al., entitled MECHANICAL FASTENING SYSTEM WITH GRIP TAB and filed Mar. 6, 1996 (attorney docket No. 12,563) which corresponds to U.S. Pat. No. 5,624,429 which issued Apr. 29, 1997, the entire disclosure of which is hereby incorporated by reference in a manner which is consistent herewith.

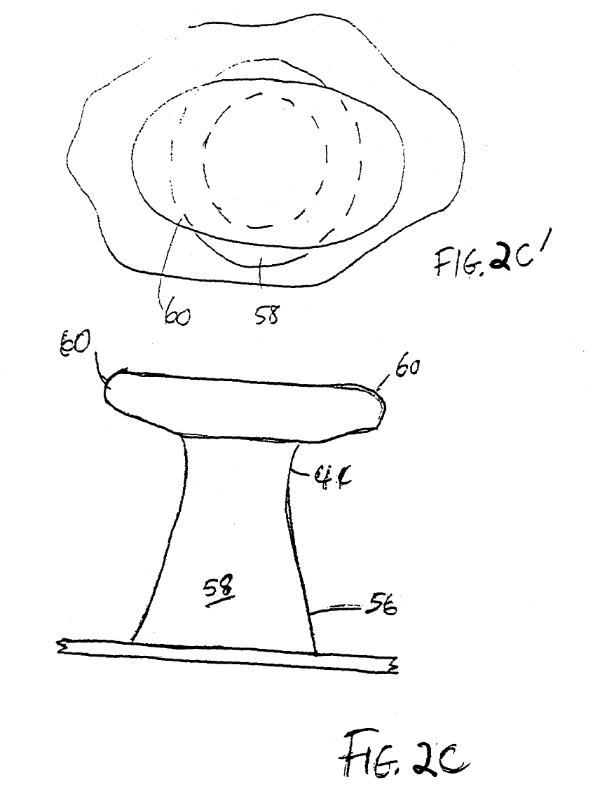

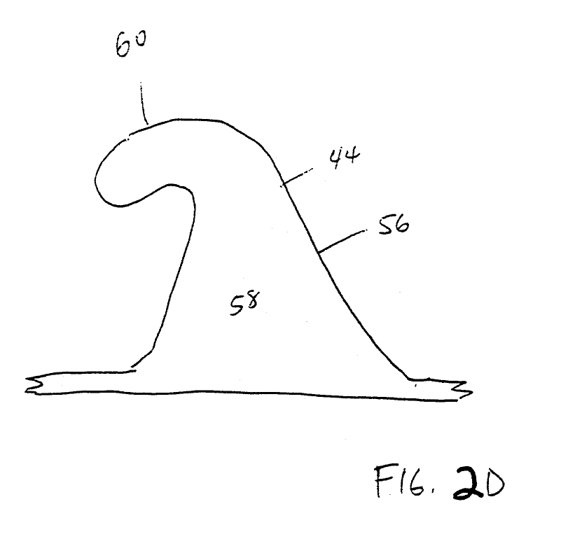

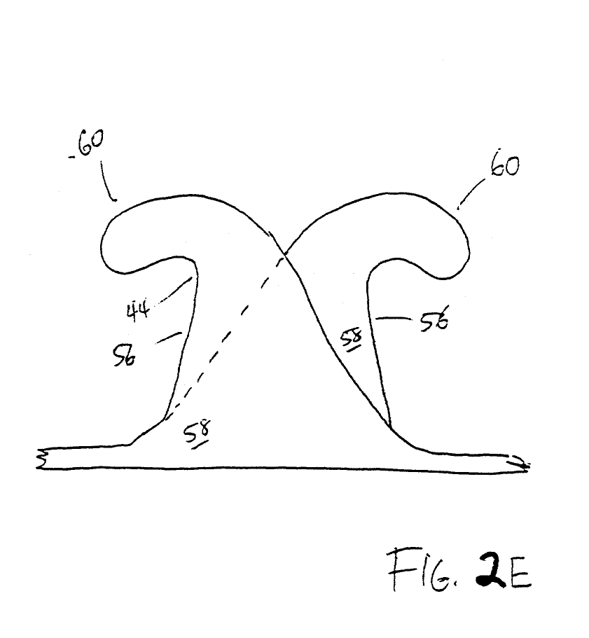

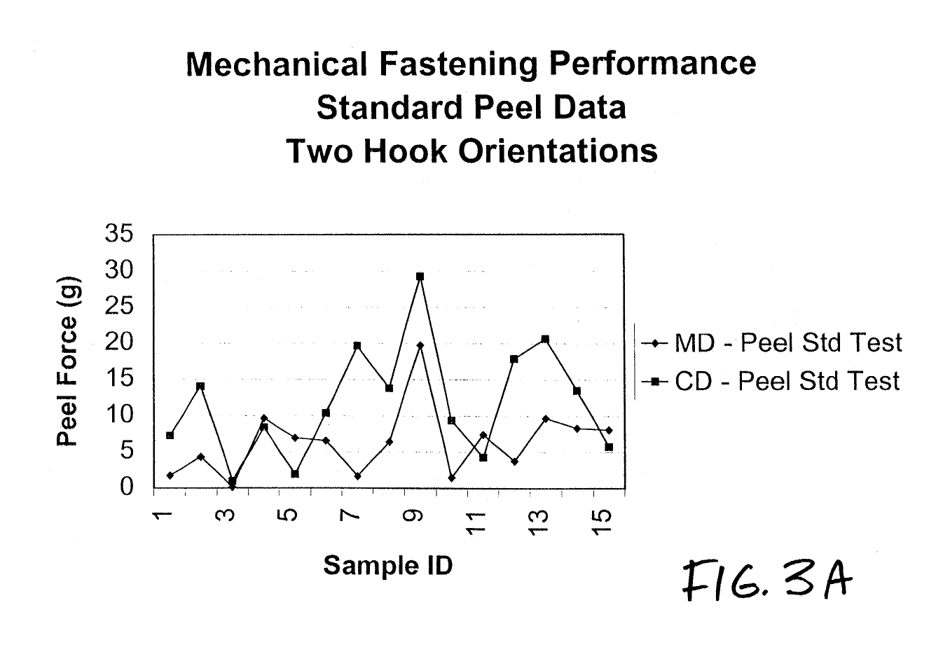

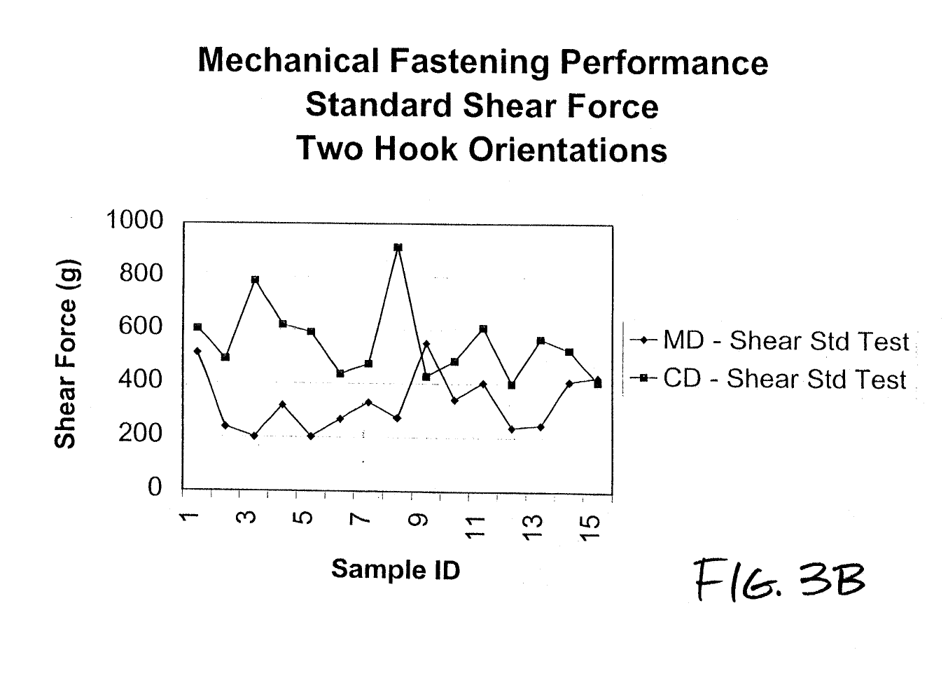

With reference to FIGS. 2A-E, the appointed first fastener component 70 can include a material having engagement members (e.g. the shown hook members) which project away from a base or substrate layer 110. Each engagement member includes a generally, up-standing stem portion 58 and a securement element 60. The stem portion 58 has a fixed end region 43, and a distal end region 44 which, desirably, is contiguously joined with the fixed end region. The fixed end region of the stem portion is operably attached to the substrate layer 110, and the distal end region is operably attached to its corresponding, associated securement element 60. The stem portion 58 is sufficiently rigid to maintain the appointed upright positioning and the appointed directional alignment of the securement element 60 during the ordinary operation of the first fastener component in the fastener system. More particularly, the stem portion is sufficiently resistant to bending and twisting to operably maintain the desired upright positioning and directional alignment of the securement element. The substrate layer 110 has a substrate thickness 112, an engagement member surface 114, and an opposed substrate mounting surface 116. The selected engagement members are attached to the substrate layer 110, and project away from the engagement member surface 114.

As representatively shown in FIGS. 2A-E, particular aspects of the invention may incorporate non-isotropic engagement members where the non-isotropic engagement members are configured to provide a directional or direction-dependent engagement with the cooperating fastener component 72. In particular, the engagement members can exhibit at least one bias direction along which a selected fastening property, such as peel force, shear force or the like, has a relatively different value. For example, the fastening property may have at least one bias direction along which a fastening property, such as peel force, shear force or the like, has a relatively maximal value. Similarly, the engagement members can exhibit at least one bias direction along which the selected fastening property has a relatively minimal value. The direction of maximal value may or may not be substantially opposite to the direction of relatively minimal value.

Thus, the non-isotropic engagement member may provide a greater (or lesser) shear force value or peel force value depending upon the direction along which the shear force or peel force value is determined. The non-isotropic feature may be generated by various suitable mechanisms, such as a difference in shape, size dimension, contour, length of projection, angle of projection, type of material, type of coating or other treatment, surface texture, surface topography, coefficient of friction, cohesion or the like, as well combinations thereof. The non-isotropic engagement member may have a limited degree of symmetry, such as a bilateral symmetry. Suitable non-isotropic engagement members can, for example, be provided by inverted-J shaped or generally T-shaped engagement members. In contrast, substantially isotropic engagement members may be provided by mushroom shaped engagement members where the mushroom head is substantially symmetrically distributed about its upstanding stem portion and where the appointed engagement opening is similarly substantially symmetrically distributed about its upstanding stem portion.

An example of a suitable micro-hook material is distributed under the designations VELCRO HTH 829 and VELCRO HTH 851 and is available from VELCRO U.S.A., Inc., a business having offices in Manchester, N.H. The micro-hook material can have hooks in the shape of angled hook elements, and can be configured with a hook density of about 264 hooks per square centimeter (about 1700 hooks per square inch); a hook height which is within the range of about 0.030-0.063 cm (about 0.012-0.025 inch); and a hook width which is within the range of about 0.007 to 0.022 cm (about 0.003 to 0.009 inch). The hook elements are molded onto a base layer substrate having a thickness of about 0.0076-0.008 cm (about 0.003-0.0035 inch), and the member of hook material has a Gurley stiffness of about 12 mgf (about 12 Gurley units). Other suitable hook materials can include VELCRO HTH 858, VELCRO HTH 851 and VELCRO HTH 863 hook materials.

Desirably, the hook materials will be in the form of a flexible tape or strip having low levels of stiffness. The provides advantages be allowing the hook material to flex along with the flexible wing material to which it is affixed and to the loop material to which it is mechanically engaged. It also permits the hook material adjacent the fabric of the undergarment to flex and bend with the undergarment to enhance engagement and to avoid a stiff component that might be a source of discomfort to a wearer of the article.

For the purposes of the present invention, the various stiffness values are determined with respect to a bending moment produced by a force which is directed perpendicular to the plane substantially defined by the length and width of the component being tested. A suitable technique for determining the stiffness values; described herein is a Gurley Stiffness test, a description of which is set forth in TAPPI Standard Test T 543 om-94 (Bending Resistance of Paper (Gurley type tester)). A suitable testing apparatus is a Gurley Digital Stiffness Tester; Model 4171-D manufactured by Teledyne Gurley, a business having offices in Troy, N.Y. For purposes of the present description, the stated Gurley stiffness values are intended to correspond to the values that would be generated by a “standard” sized sample. Accordingly, the scale readings from the Gurley stiffness tester are appropriately converted to the stiffness of a standard size sample, and are traditionally reported in terms of milligrams of force (mgf). Currently, a standard “Gurley unit” is equal to a stiffness value of 1 mgf, and may equivalently be employed to report the Gurley stiffness.

In the various aspects and configurations of the invention, the loop material can be provided by a nonwoven, woven or knit fabric. For example, a suitable loop material fabric can be composed of a 2 bar, warp knit fabric of the type available from Guilford Mills, Inc., Greensboro, N.C. under the trade designation #34285, as well as other types of knit fabrics. Suitable loop materials are also available from the 3M Company, which has distributed a nylon woven loop under their SCOTCHMATE brand. The 3M Company has also distributed a linerless loop web with adhesive on the backside of the web, and 3M knitted loop tape.

The loop material may also include a nonwoven fabric having continuous bonded areas defining a plurality of discrete unbonded areas. The fibers or filaments within the discrete unbonded areas of the fabric are dimensionally stabilized by the continuous bonded areas that encircle or surround each unbonded area, such that no support or backing layer of film or adhesive is required. The unbonded areas are specifically designed to afford spaces between fibers or filaments within the unbonded area that remain sufficiently open or large to receive and engage hook elements of the complementary hook material. In particular, a pattern-unbonded nonwoven fabric or web may include a spunbond nonwoven web formed of single component or multi-component melt-spun filaments. At least one surface of the nonwoven fabric can include a plurality of discrete, unbonded areas surrounded or encircled by continuous bonded areas. The continuous bonded areas dimensionally stabilize the fibers or filaments forming the nonwoven web by bonding or fusing together the portions of the fibers or filaments that extend outside of the unbonded areas into the bonded areas, while leaving the fibers or filaments within the unbonded areas substantially free of bonding or fusing. The degree of bonding or fusing within the bonding areas desirably is sufficient to render the nonwoven web non-fibrous within the bonded areas, leaving the fibers, or filaments within the unbonded areas to act as “loops” for receiving and engaging hook elements.

Examples of suitable point-unbonded fabrics are described in U.S. patent application Ser. No. 754,419 entitled PATTERN-UNBONDED NONWOVEN WEB AND PROCESS FOR i15 MAKING THE SAME, by T. J. Stokes et al., and filed December 17, 1996 (attorney docket No. 12,232); the entire disclosure of which is incorporated herein by reference in a manner that is consistent herewith.

In the various configurations of the invention, the loop material need not be limited to a discrete or isolated patch on the outward surface of the article. Instead, the loop material can be provided by a substantially continuous, outer fibrous layer which is assembled, integrated or otherwise joined to extend over a predetermined surface area of the desired article. For example, the outer fibrous layer may be arranged to extend over substantially the total exposed surface area of a cloth-like outer cover employed with the article.

In the various configurations of the invention, the engagement force between the selected first fastener component and its appointed and cooperating second fastener component should be large enough and durable enough to provide an adequate securement of the article on the wearer during use. In particular arrangements, especially where there are sufficiently high levels of engagement shear force provided by the fastening system, the fastening engagement may provide a peel force value of not less than a minimum of about 40 grams-force (gmf) per inch of the “width” of engagement between the first and second fastener components. In further arrangements, the fastening engagement may provide a peel force value of not less than about 100 gmf/inch to provide improved advantages. In desired configurations, the fastening engagement may provide a peel force value of not less than about 200 gmf per inch of the “width” of engagement between the first and second fastener components. Alternatively, the peel force is not less than about 300 gmf/inch, and optionally is not less than about 400 gmf/inch to further provide improved benefits. In other aspects, the peel force is not more than about 1,200 gmf/inch. Alternatively, the peel force is not more than about 800 gmf/inch, and optionally is not more than about 600 gmf/inch to provide improved performance.

The engagement force between the selected first fastener component and its appointed and cooperating second fastener component may additionally provide a shear force value of not less than about 400 gmf per square inch of the area of engagement between the first and second fastener components. Alternatively, the shear force is not less than about 1,000 gmf/in², and optionally, is not less than about 1,700 gmf/in². In further aspects, the shear force can be up to about 4,400 gmf/in², or more. Alternatively, the shear force is not more than about 3,900 gmf/in², and optionally is not more than about 3,500 gmf/in² to provide improved performance.

The peel force value can be determined in accordance with standard procedure ASTM D-5170, approved Sept. 15, 1991 and published November 1991; with the following particulars. The test specimen is the fastener tab from the article being assessed. The test specimen length is the dimension aligned along the direction in which a peel-away force is typically applied to disengage and remove the fastener during the ordinary use of the article with which the fastener is employed. The specimen “width” lies within the general plane of the fastener and is perpendicular to the specimen length. The roller device weighs 4.5 pounds and includes a rubber coating around the roller circumference.

A suitable roller is part number HR-100 available from Chemsultants International, a business having a location in Mentor, Ohio. During the engagement of the fastener components, the roller is rolled over the test specimen through one cycle in the direction of the cross-wise “width” of the sample. In addition, the initial peel by hand to “raise the loops” is omitted. During testing, the fastener material held by the stationary clamp can be larger in area, as compared to the fastener material held in the moving clamp. The initial separation distance between the clamps of the tensile tester is 4 inch, and the extension speed of the tensile testing machine is 20 inch/min. The reported value of a peel test result is a “three-peak average” value employing MTS TESTWORKS software with a peak criteria of 2%. Additionally, the peel force value is normalized to be stated in terms of force per unit length of the “width” dimension of the fastener component on the test specimen, such as grams per inch. The MTS TESTWORKS software is available from MTS Systems Corporation, a business having offices in Eden Prairie, Minn.

The shear force value can be determined in accordance with the standard procedure ASTM D-5169, approved Sep. 15, 1991 and published November 1991 with the following particulars. The test specimen is composed of the fastener tab from the article being assessed. The test specimen length and width typically correspond to the length and width employed to conduct the testing for peel force value. Ordinarily, the test specimen length is the dimension aligned along the direction in which a shear force is typically applied to the fastener during the ordinary use of the article with which the fastener is employed. The specimen “width” lies within the general plane of the fastener and is perpendicular to the specimen length. The roller device weighs 4.5 pounds and includes a rubber coating around the roller. A suitable roller is part number HR-100 available from Chemsultants International, a business having a location in Mentor, Ohio. During the engagement of the fastener components, the roller is rolled over the test specimen through five cycles in the direction of the cross-wise “width” of the sample. In addition, the initial peel by hand to “raise the loops” is omitted. During testing, the fastener material (e.g. the loop material) held by the stationary clamp can be larger in area, as compared to the fastener material (e.g. hook material) held in the moving clamp. The initial separation distance between the clamps of the tensile tester is 4 inch, and the extension speed of the tensile testing machine is 10 inch/min. The shear force value is normalized to be stated in terms of force per unit area of the test specimen, such as grams per inch².

The particulars of the standard test procedures are intended to generate fastening conditions that can be more representative of consumer use conditions. When preparing the test specimen materials (e.g. hook and loop materials) to determine the cooperating peel and/or shear force values for the representatively shown configurations of the invention, it should be noted that, the width dimension of the selected specimen material will correspond to the dimension of the fastener material which, in the actual article, is found to be aligned along the longitudinal direction 26 of the article. Similarly, the length dimension of the selected specimen material will correspond to the dimension of the fastener material which, in the actual article, is found to be aligned along the lateral direction 24 of the article.

Desirably, the securing engagement between the first fastener component and the cooperating fastener components should be sufficient to prevent a disengagement of the components when subject to a tensile force of at least about 1,000 grams when the tensile force is applied outwardly along the lateral direction, aligned generally parallel with the plane of the backsheet layer of the article.

With respect to the engagement between the first fastener component and the fabric of the undergarment, it is generally thought that the peel strength and shear force values will generally be lower than the values measured between the first and second fastener components. While it is contemplated that the engagement between the first fastener component and the fabric of the undergarment may be greater than the engagement between the first and second fastener components, it is generally thought that the engagement may be some fraction or ratio (less than 1) of the engagement between the first and second fastener components. For example, the engagement between the first fastener component and the fabric may be more than 10 percent lower than the engagement between the first and second fastener components. As another example, the engagement may be more than 20 percent lower to about 99 percent lower. As another example, the engagement may be more than 40 percent. As yet another example, the engagement may be more than 50 percent lower. As still another example, the engagement may be more than 74 percent lower.

Each of the fastener components and fastening elements in the various constructions of the invention may be operably attached to its supporting substrate by employing any one or more of the attachment mechanisms employed to construct and hold together the various other components of the article of the invention. The fastening elements in the various fastening regions, may be integrally formed, such as by molding, co-extrusion or the like, along with their associated substrate layer. The substrate layer and its associated mechanical fastening elements may be formed from substantially the same polymer material, and there need not be a discrete step of attaching the fastening elements to an initially separate substrate layer. For example, the individual hook elements may be integrally formed simultaneously with a hook base-layer by coextruding the base layer and hook elements from substantially the same polymer material.

It should be readily appreciated that the strength of the attachment or other interconnection between the substrate layer and the attached fastening component should be greater than the peak force required to remove the fastener tab 36 from its releasable securement to the appointed landing member of the article.

### EXAMPLES

The following examples are presented to provide a more detailed understanding of the invention, and are not intended to specifically limit the scope of the invention.

Peel testing was conducted generally in accordance with ASTMID-5170-91 and shear testing was conducted generally in accordance with ASTM D-51169-91. Each test utilized 10 mm squares of HTH-851 hook material available from VELCRO hand-mounted on 2.0 osy PRESTO PUB loop material (See U.S. application Ser. No. 754,419). The hook material was oriented in either MD or CD for the hook direction. The pads were worn by a mechanical walking model for 30 minutes at 72 strides/minute.

### Equipment

Tensile Tester—Constant Rate of Extension tensile tester with MTS TestWorks software.

Walking Model (Size 5 torso)

Fruit of the Loom Women's Underwear (Size 5)

### Sample Preparation

For Standard Peel and Shear testing:

Wings are removed from the product cutting along the line of adhesive juncture.

Wings are then engaged used a mechanical roller to consistently engage wings Mechanical roller available from Cheminstruments with a 2 Kg weight.

Sample is then ready for Peel or Shear testing.

For post wear testing of Peel and Shear:

Underwear are placed on Walking Model.

Pad is placed in underwear. Edge of front lobe always 1 cm in front of the crotch seam.

Wings are then engaged and the underwear pulled onto the model.

Model is set to walk at 72 strides per minute for 30 minutes.

Underwear are cut off the model and removed carefully to not impact hook engagement.

Wings are the removed from product and panty by cutting along the adhesive juncture.

Sample is the ready for post wear Peel and Shear testing.

### Peel Test

Finger tab (area at end of wing beyond hook is placed in the upper jaw, cut edge of other wing is place in the lower jaw. Careful not to leave too much slack or pull hook from loop engagement

Test is run at the following parameters:

Crosshead speed—20 in/min

Gauge Length—3 inches

Load units—Grams

Start Measure—0.4 in

End Measure—0.9 in

Break Sensitive—110%

Slack Compensation—50 grams

Response is the averaged gram of force over the length of the peel test.

### Shear Test

Cut edge of one wing is placed in the upper jaw and cut edge of opposing wing is placed in the lower jaw. Careful not to leave too much slack or pull hook from loop engagement.

Test is run at the following parameters:

Crosshead speed—20 in/min

Gauge Length—3 inches

Load units—Grams

Start Measure—0.4 in

End Measure—0.9 in

Break Sensitive—110%

Slack Compensation—50 grams

Response is the averaged gram of force over the length of the peel test.

Results of testing is reported in Table 1.

TABLE 1

| | MD- | CD- | MD- | CD- | MD- | CD- | MD- | CD- |
|---|---|---|---|---|---|---|---|---|
| | Peel | Peel | Shear | Shear | Peel | Peel | Shear | Shear |
| | Std | Std | Std | Std | Post | Post | Post | Post |
| | Test | Test | Test | Test | Wear | Wear | Wear | Wear |
| | 1.71 | 7.22 | 512.9 | 603.8 | 74.7 | 32.96 | 437 | 674 |
| | 4.27 | 14.02 | 237.9 | 491 | 35.92 | 12.2 | 635.8 | 649.6 |
| | 0.1 | 0.94 | 201 | 785.2 | 42.57 | 15.8 | 469.7 | 454.4 |
| | 9.65 | 8.38 | 318 | 618.8 | 36.86 | 23.16 | 1125.7 | 551.4 |
| | 6.9 | 1.88 | 202 | 591.9 | 206.03 | 26.26 | 1208.8 | 485.6 |
| | 6.53 | 10.37 | 267.9 | 435 | 34.78 | 23.49 | 510.8 | 519.7 |
| | 1.61 | 19.63 | 329.9 | 473 | 72.98 | 13.74 | 685.3 | 455.8 |
| | 6.39 | 13.8 | 272.7 | 912.7 | 57.75 | 33.29 | 566.7 | 554.7 |
| | 19.72 | 29.26 | 550.8 | 427.2 | 78.67 | 25.35 | 780.9 | 581 |
| | 1.46 | 9.38 | 341.4 | 484.4 | 67.47 | 16.29 | 934.5 | 383.1 |
| | 7.42 | 4.24 | 403.7 | 610.4 | 54.37 | 43.95 | 382.3 | 466.6 |
| | 3.75 | 17.86 | 237.4 | 400.4 | 54.5 | 29.39 | 523.9 | 434.5 |
| | 9.69 | 20.62 | 246.4 | 567.5 | 66.31 | 32.2 | 740.1 | 299.5 |
| | 8.31 | 13.53 | 408.3 | 525.6 | 23.65 | | 619.1 | 438.7 |
| | 8.07 | 5.74 | 424.2 | 407.3 | 47.73 | | 771.5 | 425.9 |
| Average | 6.4 | 11.8 | 330.3 | 555.6 | 63.6 | 25.2 | 692.8 | 491.6 |
| Std | 4.8 | 7.8 | 109.3 | 142.7 | 42.7 | 9.2 | 242.9 | 98.9 |
| Dev. | | | | | | | | |

The data are summarized in Table 2. For both shear and peel values, the orientation having the lowest weakest initial MD and CD peel and shear strength values (i.e., the ‘illogical’ orientation) engaged strongly during use. In contrast, the orientation having the greatest or strongest initial MD and CD peel and shear strength values (i.e., the “logical” orientation) either strengthens somewhat during wear or falls in strength. Also, for both the shear and peel value, the absolute after wear values are higher for the ‘illogical’ orientation than for the ‘logical’.

TABLE 2

| | | | after | | ratio, before |
|---|---|---|---|---|---|
| | before wear | SD | wear | SD | to after |
| illogical (MD) peel | 6.4 | 4.8 | 64 | 43 | 10.0 |
| logical (CD) peel | 11.8 | 7.8 | 25 | 9 | 2.1 |
| illogical (MD) shear | 330 | 109 | 693 | 242 | 2.1 |
| logical (CD) shear | 556 | 143 | 491 | 99 | 0.9 |

This phenomena is thought to also occur for those first fastener components or “hook components” that engage the fabric of an undergarment.

These examples illustrate an embodiment of the present invention in which an absorbent article such as, for example, a sanitary napkin has an attachment system that includes a pair of wings including selectively releasable, interengaging fasteners such that the wings are adapted to hold the sanitary napkin to an undergarment. The wings are adapted to increase engagement during use. In addition, the attachment system includes a fastener component including a plurality of engagement members so it is adapted to engage the fabric of an undergarment. This fastener component may also be adapted to engage and/or increase engagement with the fabric of an undergarment during use.

Having described the invention in rather full detail, it will be readily apparent that various changes and modifications can be made without departing from the spirit of the invention.

We claim:

1. A sanitary napkin having a pair of end edges, a first longitudinal edge and a second longitudinal edge located between the end edges, a backsheet layer, a substantially liquid permeable topsheet layer and an absorbent disposed between the 5 topsheet layer and the backsheet layer; the sanitary napkin including a selectively releasable, interengaging fastening system comprising:
a first wing extending from the first longitudinal edge of the sanitary napkin and a second wing extending from the second longitudinal edge of the sanitary napkin, each wing having a fixed end and a free end;
a first fastener component comprising at least a portion of the first wing and the second wing, the first fastener component including an engagement section having a plurality engagement members; and
a cooperating fastener component comprising at least a portion of the first wing and the second wing such that the first and second wings of the sanitary napkin are capable of being joined by movement of the respective components together generally along an attachment direction into an overlapping and interengaging configuration;
wherein the first fastener component is configured on the first wing and the second wing so that the overlapped and interengaged wings are adapted to secure the sanitary napkin to an undergarment, and an unengaged first fastener component is positioned adjacent the backsheet so it is adapted to engage the fabric of an undergarment positioned between the backsheet and the wings.

2. The sanitary napkin of claim 1, wherein the first fastener component has a plurality of substantially non-isotropic engagement members such that the engagement section has an axis of substantially maximal engagement and the first fastener component is oriented so its axis of substantially maximal engagement is generally orthogonal to the attachment direction.

3. The sanitary napkin of claim 2, wherein each substantially non-isotropic engagement member has a stem portion with a distal end region and a securement element disposed at the distal end region of its corresponding stem portion.

4. The sanitary napkin of claim 1, wherein the second fastener component is a nonwoven loop material.

5. The sanitary napkin of claim 4, wherein the nonwoven loop material is a pattern unbonded material.

6. The sanitary napkin of claim 1, wherein the fastening system can be used to secure the sanitary napkin in a configuration convenient for disposal.

7. The sanitary napkin of claim 1, further comprising garment attachment adhesive on the backsheet layer.

8. The sanitary napkin of claim 1, wherein at least one of the first and second wings is substantially liquid permeable.

9. The sanitary napkin of claim 1, wherein at least one of the first and second wings is substantially liquid impermeable.

10. A multiple attachment system for a sanitary napkin having a pair of end edges, a first longitudinal edge and a second longitudinal edge located between the end edges, a backsheet layer, a substantially liquid permeable topsheet layer and an absorbent disposed between the topsheet layer and the backsheet layer; multiple attachment system including a selectively releasable, interengaging fastening system comprising:
a first wing extending from the first longitudinal edge of the sanitary napkin and a second wing extending from the second longitudinal edge of the sanitary napkin, each wing having a fixed end and a free end;
a first fastener component comprising at least a portion of the first wing and the second wing, the first fastener component including an engagement section having a plurality of engagement members; and
a cooperating fastener component comprising at least a portion of the first wing and the second wing such that the first and second wings of the sanitary napkin are capable of being joined by movement of the respective components together generally along an attachment direction into an overlapping and interengaging configuration;
wherein the first fastener component is configured on the first wing and the second wing so the overlapped and interengaged wings are adapted to secure the sanitary napkin to an undergarment, and further present an unengaged first fastener component adjacent the backsheet so it is adapted to engage the fabric of an undergarment positioned between the backsheet and the wings.

11. The multiple attachment system of claim 10, wherein the first fastener component has a plurality of substantially non-isotropic engagement members such that the engagement section has an axis of substantially maximal engagement and the first fastener component is oriented so its axis of substantially maximal engagement is generally orthogonal to the attachment direction.

12. The multiple attachment system of claim 10, wherein each substantially non-isotropic engagement member has a stem portion with a distal end region and a securement element disposed at the distal end region of its corresponding stem portion.

13. The multiple attachment system of claim 10, wherein the second fastener component is a nonwoven loop material.

14. The multiple attachment system of claim 13, wherein the nonwoven loop material is a pattern unbonded material.

15. The multiple attachment system of claim 10, wherein the attachment system can be used to secure the sanitary napkin in a configuration convenient for disposal.

16. The multiple attachment system of claim 10, further comprising garment attachment adhesive on the backsheet layer.

17. A sanitary napkin having a pair of end edges, a first longitudinal edge and a second longitudinal edge located between the end edges, a backsheet layer, a substantially liquid permeable topsheet layer and an absorbent disposed between the topsheet layer and the backsheet layer; the sanitary napkin including a multiple attachment system comprising:
a first wing extending from the first longitudinal edge of the sanitary napkin and a second wing extending from the second longitudinal edge of the sanitary napkin, each wing having a fixed end and a free end;
a first fastener component comprising at least a portion of a bottom surface of the first wing and the second wing, the first fastener component including an engagement section having a plurality of engagement members; and
a cooperating fastener component comprising at least a portion of a top surface the first wing and the second wing such that the first and second wings can be interengaged by bringing a top and bottom surface of respective wings into an overlapping configuration;
wherein the interengaged wings are adapted to secure the sanitary napkin to an undergarment, and further present an unengaged first fastener component adjacent the backsheet so it is adapted to engage the fabric of an undergarment positioned between the backsheet and the wings.

18. The sanitary napkin of claim 17, wherein the wings of the sanitary napkin can be moved together into their overlapping configuration generally along an attachment direction.

19. The sanitary napkin of claim 17, wherein the first fastener component has a plurality of substantially non-isotropic engagement members such that the engagement section has an axis of substantially maximal engagement.

20. The sanitary napkin of claim 19, wherein the first fastener component is oriented so its axis of substantially maximal engagement is generally orthogonal to the attachment direction.

21. The sanitary napkin of claim 19, wherein each substantially non-isotropic engagement member has a stem portion with a distal end region and a securement element disposed at the distal end region of its corresponding stem portion.

22. The sanitary napkin of claim 17, wherein the second fastener component is a nonwoven loop material.

23. The sanitary napkin of claim 22, wherein the nonwoven loop material is a pattern unbonded material.

24. The sanitary napkin of claim 17, wherein the attachment system can be is used to secure the sanitary napkin in a configuration convenient for disposal.

25. The sanitary napkin of claim 17, further comprising garment attachment adhesive on the backsheet layer.

26. The sanitary napkin of claim 17, wherein at least one wing includes perforation lines adjacent its fixed end to provide quick removal of the napkin by tearing the wing along the perforation lines.

27. A sanitary napkin having a pair of end edges, a first longitudinal edge and a second longitudinal edge located between the end edges, a backsheet layer, a substantially liquid permeable topsheet layer and an absorbent disposed between the topsheet layer and the backsheet layer;
the sanitary napkin further including a pair of wings, each wing extending a longitudinal edge of the sanitary napkin and each wing having a fixed end and a free end; each wing comprising:
a first fastener component on a lower surface of the wing, the first fastener component including an engagement section having a plurality engagement members; and
a cooperating fastener component on an upper surface of the wing such that the wings of the sanitary napkin are capable of being joined by bringing the top and bottom surface of respective wings into an overlapping and interengaging configuration;
wherein the interengaged wings are adapted to secure the sanitary napkin to an undergarment and further present an unengaged first fastener component adjacent the backsheet so it is adapted to engage the fabric of an undergarment positioned between the backsheet and the wings.

28. A sanitary napkin including an attachment system comprising:
a pair of wings including selectively releasable, interengaging fasteners such that the wings are adapted to hold the sanitary napkin to an undergarment; and
a fastener component including a plurality of engagement members so it is adapted to engage the fabric of an undergarment.
